(19) United States
(12) Patent Application Publication
(10) Pub. No.: US 2019/0134121 A1
(43) Pub. Date: May 9, 2019

(54) METHOD FOR REDUCTION, SUPPRESSION, OR ELIMINATION OF ANXIETY OR MARIJUANA/CANNABIS EFFECTS AND RELATED MARIJUANA/CANNABIS PRODUCT BY PROCESS

(71) Applicants: Steven Bermudez, Santa Barbara, CA (US); Aaron Brown, Las Vegas, NV (US)

(72) Inventors: Steven Bermudez, Santa Barbara, CA (US); Aaron Brown, Las Vegas, NV (US)

(21) Appl. No.: 16/059,002

(22) Filed: Aug. 8, 2018

Related U.S. Application Data
(60) Provisional application No. 62/542,617, filed on Aug. 8, 2017.

(51) Int. Cl. A61K 36/185; A61K 31/05; A61K 31/136; A61K 31/522; A61K 9/00; A61K 31/192

(52) U.S. Cl. CPC A61K 36/185; A61K 31/05; A61K 31/192; A61K 31/522; A61K 9/006; A61K 31/136

(57) ABSTRACT

In one aspect, a formulation comprising cannabinoids, paracetamol, methylxanthines, salicylates, terpenes, humulus oil, or amino acids individually or any combination or omission thereof for the reduction, alleviation, elimination, and/or suspension of effects of THC exposure and anxiety. The formulation can be the effective amount of cannabinoid is between 5 mg and 5000 mg. The formulation can be the cannabinoid comprises a Cannabidiol (CBD), a cannabidolic acid (CBDA), a Cannabinol (CBN), a Cannabigerol (CBG), a Cannabichromene (CBC), a Cannabicyclol (CBL), a Cannabivarin (CBV), a Tetrahydrocannabivarin (THCV), a Cannabidivarin (CBDV), a Cannabichromevarin (CBCV), a Cannabigerovarin (CBGV), a Cannabigerol monomethyl ether (CBGM), a Cannabielsoin (CBE), or a cannabicitran (CBT). The formulation can be the effective amount of paracetamol is between 0 mg-1000 mg.

### CLAIM OF PRIORITY

This application claims priority from U.S. Provisional Application No. 62/542,617, METHOD FOR SUPPRESSION OF MARIJUANA EFFECTS AND RELATED PRODUCT BY PROCESS and filed 8 Aug. 2017. This application is hereby incorporated by reference in its entirety for all purposes.

### FIELD OF THE INVENTION

This description relates to the field of using any number of synthetic or naturally occurring cannabinoids, cannabinoid derivatives, paracetamols, methylxanthines, and salicylates alone or in any combination for the purpose of alleviating, reducing, suppressing, or eliminating the intoxicating effects of THC and of anxiety.

### DESCRIPTION OF THE RELATED ART

Cannabinoids may serve as a direct or indirect modulator of the endocannabinoid system resulting in both psychological and physiological effects. These psychological and physiological effects can have a deleterious effect on a user's lifestyle and productivity. Accordingly, products that relieve these psychological and physiological effects are desired.

### SUMMARY

In one aspect, a formulation comprising cannabinoids, paracetamol, methylxanthines, salicylates, terpenes, humulus oil, or amino acids individually or any combination or omission thereof for the reduction, alleviation, elimination, and/or suspension of effects of THC exposure and anxiety. The formulation can be the effective amount of cannabinoid is between 5 mg and 5000 mg. The formulation can be the cannabinoid comprises a Cannabidiol (CBD), a cannabidolic acid (CBDA), a Cannabinol (CBN), a Cannabigerol (CBG), a Cannabichromene (CBC), a Cannabicyclol (CBL), a Cannabivarin (CBV), a Tetrahydrocannabivarin (THCV), a Cannabidivarin (CBDV), a Cannabichromevarin (CBCV), a Cannabigerovarin (CBGV), a Cannabigerol monomethyl ether (CBGM), a Cannabielsoin (CBE), or a cannabicitran (CBT). The formulation can be the effective amount of paracetamol is between 0 mg-1000 mg. The formulation can be the effective amount of methylxanthines is between 0 mg-1000 mg. The formulation can be the methylxanthine comprises caffeine, aminophylline, paraxanthine, pentoxifylline, 3-isobutyl-1-methylxanthine, theobromine, or theophylline. The formulation can be the effective amount of salicylic acid or derivatives of salicylic acid are between 0 mg and 1000 mg. The formulation can be the salicylic acid or derivatives of salicylic acid comprises methylsalicylic acid, benzoic acid, phenol, 4-hydroxybenzoic acid, acetylsalicylic acid, magnesium salicylate, choline salicylate, bismuth subsalicylate, or sulfosalicylic acid. The formulation can be the terpene comprises alpha pinene, linalool, beta-caryophyllene, myrcene, limonene, ocimene, terpinolene, terpineol, valencene, geraniol, humulene, phellandrene, careen, terpinene, fenchol, borneol, bisabolol, phytol, camphene, sabinene, camphor, isoborneol, menthol, cedrane, nerolidol, guaiol, isopulegol, geranyl acetate, cymene, eucalyptol, or pulegone. The formulation can be the effective amount of terpene is between 0.25 mg-1000 mg. The formulation can be the effective amount of amino acid is between 1 mg/1000 mg. The formulation can be the amino acids comprises theanine or GABA. The formulation can be the formulation comprises a suspension in a liquid emulsification suitable for oral application via a pump spray. The formulation can be the formulation comprises the suspension is suitable for a softgel, a tablet, a capsule, a gummie, a lozenge, a chewing gum, a taffy, a vape liquid, an inhaler cartridge, an aerosol, a topical ointment, a tincture, a transdermal patch, a suppository, or an edible product.

### BRIEF DESCRIPTION OF THE DRAWINGS

FIG. 1 illustrates an example process for suppressing the effects of marijuana or cannabis with a Cannabidiol, hemp oil, hemp oil extracts, cannabis oil, cannabis extracts, terpenes, humulus (extract) and/or humulus oil-based formulation, according to some embodiments.

FIG. 2 illustrates an example process for manufacturing a Cannabidiol, hemp oil, cannabis oil, terpene, and/or humulus oil-based formulation, according to some embodiments. provide an emulsion, according to some embodiments.

FIG. 3 illustrates an example oil in water suspension, according to some embodiments.

FIG. 4 illustrates an example water in oil suspension, according to some embodiments.

FIG. 5 Illustrates an example 10 ml solution, according to some embodiments.

FIG. 6 illustrates an example water in Oil suspension, according to some embodiments.

FIG. 7 illustrates an example Medium-chain triglycerides (MCT) oil base without GABA and I-theanine Alpha and humulus oil in place of CBD, according to some embodiments.

The Figures described above are a representative set and are not an exhaustive with respect to embodying the invention.

### DESCRIPTION

Disclosed are a system, method, and article of manufacture for reduction, suppression, or elimination of anxiety and/or Marijuana/cannabis effects. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to ‘one embodiment,’ ‘an embodiment,’ ‘one example,’ or similar language means that a particular feature, structure, or characteristic described in connection with the embodiment is included in at least one embodiment of the present invention. Thus, appearances of the phrases ‘in one embodiment,’ ‘in an embodiment,’ and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Definitions

Example definitions for some embodiments are now provided.

Adenosine is a purine nucleoside composed of a molecule of adenine attached to a ribose sugar molecule (ribofuranose) moiety via a β-N9-glycosidic bond.

Cannabinoid can be one of a class of diverse chemical compounds that acts on cannabinoid receptors in cells that alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially). An example cannabinoid is the phytocannabinoid tetrahydrocannabinol (THC), the primary psychoactive compound in cannabis. A cannabinoid can include the following chemicals: Tetrahydrocannabinol (THC), tetrahydrocannabinolic acid (THCA), iso-tetrahydrocannabinol (iso-THC), iso-tetrahydrocannabinolic acid (Iso-THCA), cannabidolic acid (CBDA) Cannabinol (CBN), Cannabigerol (CBG), Cannabichromene (CBC), Cannabicyclol (CBL) Cannabivarin (CBV), Tetrahydrocannabivarin (THCV), Cannabidivarin (CBDV), Cannabichromevarin (CBCV), Cannabigerovarin (CBGV), Cannabigerol monomethyl ether (CBGM), Cannabielsoin (CBE), and cannabicitran (CBT).

Cannabidiol (CBD) is one of many active cannabinoids identified in cannabis. Cannabidiol may have effects on anxiety and an anti-psychotic effect. Derived from multiple natural and synthetic sources, including but not limited to cannabis, industrial hemp, hops, lichen moss, citrus refuse (e.g. a lemon peel, etc.), humulus, etc.

Emulsion is a mixture of two or more liquids that are normally immiscible (e.g. unmixable or unblendable).

Ethanol is the principal type of alcohol found in alcoholic beverages. It is a volatile, flammable, colorless liquid with a slight characteristic odor. Its chemical formula is C2H6O, which can be written also as CH3-CH2-OH or C2H5-OH (an ethyl group linked to a hydroxyl group), and is often abbreviated as EtOH.

Haematopoiesis is the formation of blood cellular components.

Paracetamol (acetaminophen or APAP) is a medication used to treat pain and fever.

Prostaglandins (PG) are a group of physiologically active lipid compounds having diverse hormone-like effects in animals. PG are derived enzymatically from fatty acids. Every prostaglandin contains twenty (20) carbon atoms, including a 5-carbon ring. PG are a subclass of eicosanoids and of the prostanoid class of fatty acid derivatives.

L-Theanine—is a phytonutrient amino acid analog of glutamate commonly found in green tea and gyokuro leaves known for its stimulatory effects in the brain. Several studies have concluded that L-Theanine enhances visual attention and reaction time while reducing heart rate. Additionally, a recent clinical trial review concluded that L-Theanine significantly reduced acute stress and anxiety amongst study participants placed in stressful situations.

GABA—Gamma Aminobutyric acid—is an inhibitory neurotransmitter known to relieve anxiety and improve focus. Although neuroscientists have studied GABA for years in brain function and development, only recently has it been recognized as a viable supplement capable of stimulating the enteric nervous system to produce endogenous GABA and cross the blood brain barrier. There has been recent interest in GABA as a dietary supplement and many anti-psychotic drugs are synthetic analogs of GABA.

Alpha Pinene is a terpene and is a common phytonutrient found in several plant and tree species, including pine (where it derived its name). Although the anti-inflammatory and antioxidant health benefits of alpha pinene have been studied since the mid-1950's, there has been recent interest in how this molecule can counter the memory deficit effects of THC induced intoxication.

Beta Pinene is a monoterpene, an organic compound found in plants. It is one of the two isomers of pinene, the other being α-pinene.

Linalool is a terpene found in over two-hundred (200) species of plants and trees and is commonly used as a fragrance for its calming effects. Linalool has been shown to have anxiolytic effects in several animal and human studies. Furthermore, it has been shown to suppress aggressive behavior and enhance social interaction in several animal studies.

Beta Caryophyllene can be a terpene that has been shown to provide mental and health benefits including anti-inflammatory, antioxidant, and neuroprotective properties. Additionally, recent studies have shown beta caryophyllene has anti-depressive and anti-anxiety properties. But one of the most interesting studies has recently shown that this molecule can bind and modify the cannabinoid receptors, indicating a potential mechanism of detoxification.

Hemp Oil—Industrial Hemp is known for its high Cannabidiol (e.g. CBD, non-hallucinogenic cannabinoid) content and negligible THC component (e.g. less than 0.3%). Therefore, the oils extracted from industrial hemp contain a full spectrum of terpenes and phytonutrients that provide a natural source of healthy molecules without the intoxicating effects of marijuana.

Humulus oil can be an oil derived from the humulus plant. Humulus is a variety of hop, commonly found in the Silk Road region of India. Although not all hop plants traditionally contain cannabinoids, there are particular varieties of humulus have high concentrations of cannabinoids including Cannabidiol.

Olivetol is a naturally occurring organic compound. It is found in certain species of lichens and can be readily extracted. 5-Pentylresorcinol is also produced by a number of insects, either as a pheromone, repellent or antiseptic. The cannabis plant internally produces the related substance olivetolic acid (OLA), which was hypothesized that the plant in turn utilizes to biosynthesize the psychoactive product tetrahydrocannabinol (THC).

Oil of Wintergreen include methyl salicylate (oil of wintergreen or wintergreen oil) and can be an organic compound with the formula C6H4(OH)(CO2CH3). It is the methyl ester of salicylic acid. It is a colorless, viscous liquid with a sweet odor. It is produced by many species of plants, particularly wintergreens. It is also synthetically produced, used as a fragrance, in foods and beverages, and in liniments.

Limonene is a clear, colorless liquid hydrocarbon classified as a cyclic monoterpene, and is the major component in the oil of citrus fruit peels.

Phellandrene is the name for a pair of organic compounds that have a similar molecular structure and similar chemical properties. α-Phellandrene and β-phellandrene are cyclic monoterpenes and are double-bond isomers. In α-phellandrene, both double bonds are endocyclic and in β-phellandrene, one of them is exocyclic. Both are insoluble in water, but miscible with ether.

### EXAMPLE EMBODIMENTS AND METHODS

A combination of one cannabinoid, Cannabidiol (CBD) or its acid derivative, alone or in combination with paracetamol and methylxanthine, can be used significantly reduce, eliminate, suppress, or alleviate the intoxicating effects of THC use. In this way, symptoms of marijuana or cannabis use can be reduced, eliminated, and/or suppressed by the oral, sublingual, topical, or inhalation application of a formulation.

The formulation suppresses the effects marijuana or cannabis consumption. The effective composition is an emulsion containing 0-1000 mg/ml of CBD, a prostaglandin inhibitor and a methylxanthine. These natural compounds have a combine effect to counter or work against the effect of excess THC and/or excess alcohol.

Cannabis/marijuana affects receptors mostly in the brain, but also located in the immune system, the hemopoetic system, the liver and the heart. The physiology of cannabinoid effects on humans is based on unique and specific receptors for Cannabinoid compounds that are located in the cerebral foci that modify appetite, pain sensation, mood and memory. These receptors are neuromodulatory receptors located in the cell membrane.

The formulation works by working on several cascades of receptors and inflammatory cascades. One hemp derived compound works as a competitive binding to strong THC effects. This competitive binding or inhibition can bind to the receptors and push the active Cannabis from the receptors. Symptoms of excess Cannabis or marijuana can decrease accordingly. When an individual has circulating strong THC and other Cannabinoid chemicals in their system from excess intake, the formulation can offset and bind to these Cannabinoid receptors to decrease the symptoms of excess Cannabis use. There is also an effect of the formulation on the receptors for Cannabis. By controlling or inhibiting the number of receptors for Cannabis, the effects of the THC can also be decreased.

In addition, the product works on the prostaglandin cascade. The prostaglandin effect increases the inflammation of the body. By suppressing the prostaglandin effects, pain and other inflammatory conditions go away. The prostaglandin cascade is crucial in many steps of inflammation in the body and in the brain. Because of the specific prostaglandin inhibition, the brain fog and headaches can be well-alleviated by this mechanism.

The other action of the formulation is acting on adenosine receptors of the brain and kidney. This mechanism of action is by methylxanthine compounds. These cause increase energy and increased awareness by dilating blood vessels and increasing oxygen.

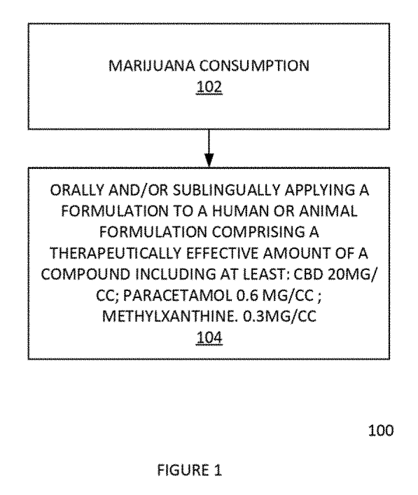

FIG. 1 illustrates an example process 100 for suppressing the effect of marijuana with a formulation, according to some embodiments. In one example, in step 102, marijuana consumption occurs. In step 104, an individual can orally and/or sublingually apply a formulation comprising a therapeutically effective amount of a formula. It is noted that this formulation is by way of example and not limitation. In other example embodiments, other ranges of the active ingredients can be utilized. For example, 5-20 mg/ml of cannabidiol can be utilized. In another example, 1-20 mg/ml of cannabidiol can be utilized. These examples are provided by way of illustration and not of limitation.

The formulation can consist of any combination of water, short carbon chain alcohols, polyethylene glycol, propylene glycol, glycerin, mineral oil, xanthan gum, citric acid, cocoa, vegetable glycerin, hemp seed oil, humulus oil, terpenes, polysorbate, polysorbate 80, natural flavors, artificial flavors, and mixtures thereof. Some of the ingredients may be omitted in certain combinations.

In one example embodiment, the formulation can be an emulsion that is safe for human consumption. For example, the formulation can be an oil-based pharmaceutical formulation. The formulation can be a tincture, liquid, oral spray, oral mist, vape liquid, inhaler cartridge, soft gel, aerosol, topical cream, transdermal patch, or in a tablet pill-form.

In one example embodiment, the formulation can be an emulsion that is safe and in a form for human sublingual delivery.

It is noted that concentrations/amounts CBD, Paracetamol and/or Methylxanthine can be varied in other example embodiments. It is also noted that the prostaglandin inhibitor can include aspirin, naproxen and/or cox-2 inhibitors in various embodiments. The Methylxanthines can include caffeine, aminophylline, and/or theophylline in various embodiments.

In some alternate embodiments, the oil-based formulation can be administered transcutaneously. In this case, the application of the formulation can be by way of a patch, cloth, direct contact, etc.

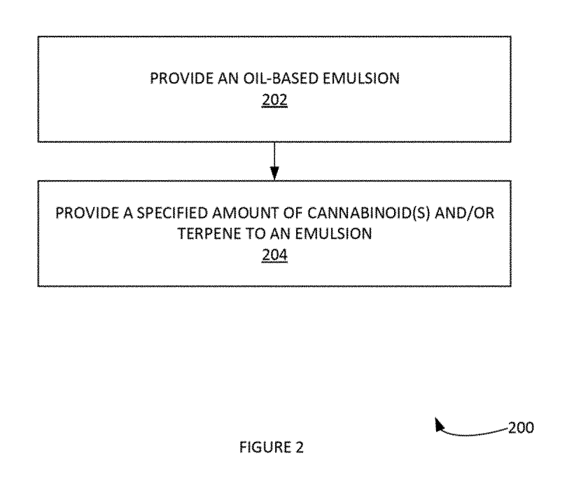

FIG. 2 illustrates an example process 200 for manufacturing a CBD-based formulation, according to some embodiments. Process 200 can provide an oil-based emulsion, according to some embodiments. In step 202, a specified amount of Cannabidiol to an oil-based emulsion can be provided.

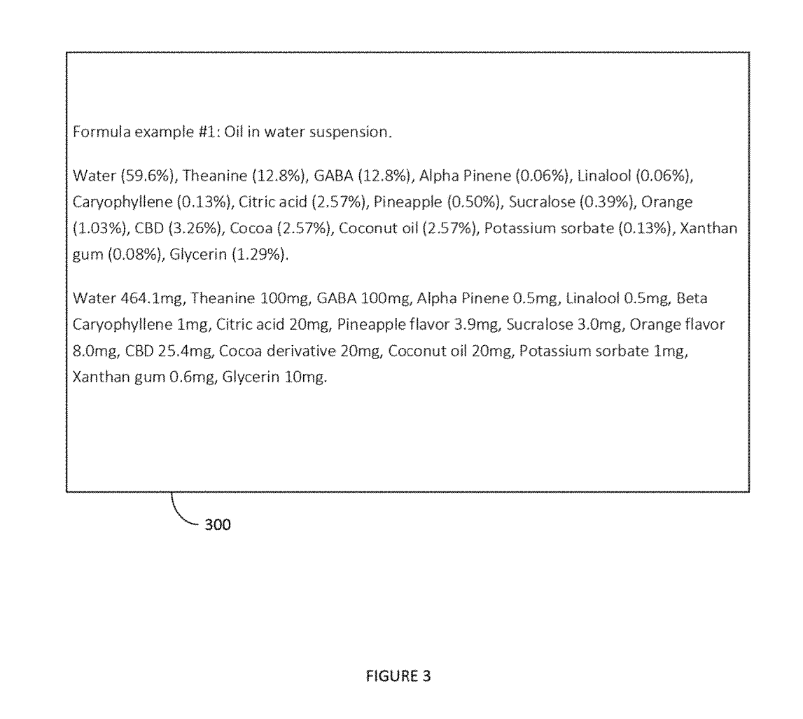

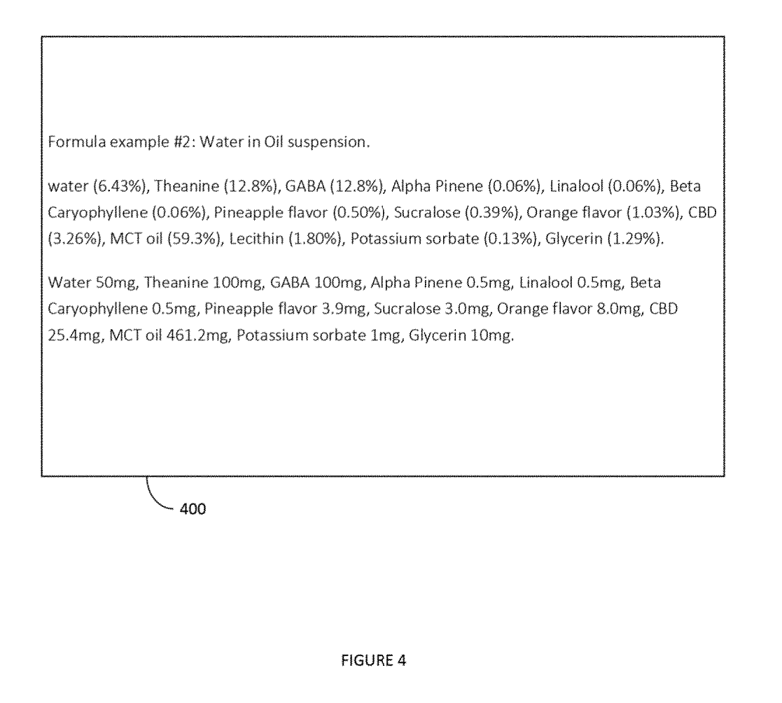

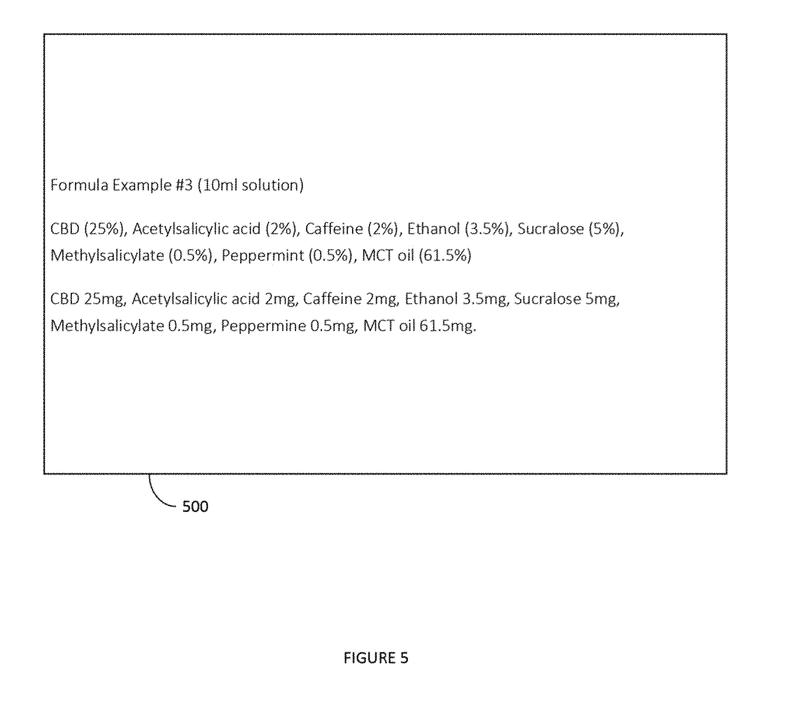

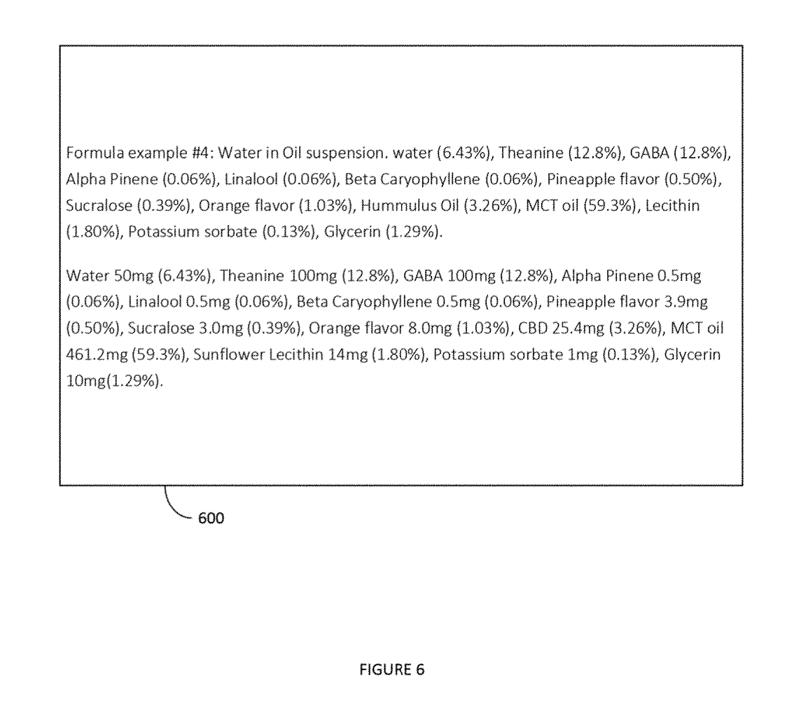

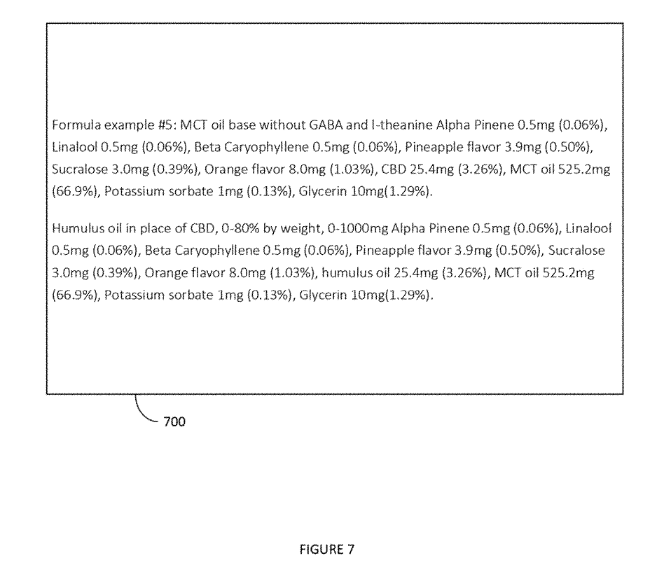

FIG. 3-5 illustrate example formulas that can be utilized. It is noted that these formulas are provided by way of example and various permutations can be utilized with various ranges of ingredients. More specifically, FIG. 3 illustrates an example oil in water suspension 300, according to some embodiments. FIG. 4 illustrates an example water in oil suspension 400, according to some embodiments. Figure S illustrates an example 10 ml solution 500, according to some embodiments. FIG. 6 illustrates an example water in Oil suspension 600, according to some embodiments. FIG. 7 illustrates an example Medium-chain triglycerides (MCT) oil base without GABA and I-theanine Alpha and humulus oil in place of CBD 700, according to some embodiments.

### CONCLUSION

Although the present embodiments have been described with reference to specific example embodiments, various modifications and changes can be made to these embodiments without departing from the broader spirit and scope of the various embodiments.

What is claimed as new and desired to be protected by Letters Patent of the United States is:

1. A formulation comprising cannabinoids, paracetamol, methylxanthines, salicylates, terpenes, humulus oil, or amino acids individually or any combination or omission thereof for the reduction, alleviation, elimination, and/or suspension of effects of THC exposure and anxiety.

2. The formulation of claim 1, wherein the effective amount of cannabinoid is between 5 mg and 5000 mg.

3. The formulation of claim 2, wherein the cannabinoid comprises a Cannabidiol (CBD), a cannabidolic acid (CBDA), a Cannabinol (CBN), a Cannabigerol (CBG), a Cannabichromene (CBC), a Cannabicyclol (CBL), a Cannabivarin (CBV), a Tetrahydrocannabivarin (THCV), a Cannabidivarin (CBDV), a Cannabichromevarin (CBCV), a Cannabigerovarin (CBGV), a Cannabigerol monomethyl ether (CBGM), a Cannabielsoin (CBE), or a cannabicitran (CBT).

4. The formulation of claim 1, wherein the effective amount of paracetamol is between 0 mg-1000 mg.

5. The formulation of claim 1, wherein the effective amount of methylxanthines is between 0 mg-1000 mg.

6. The formulation of claim 5, wherein the methylxanthine comprises caffeine, aminophylline, paraxanthine, pentoxifylline, 3-isobutyl-1-methylxanthine, theobromine, or theophylline.

7. The formulation of claim 1, wherein the effective amount of salicylic acid or derivatives of salicylic acid are between 0 mg and 1000 mg.

8. The formulation of claim 7, wherein the salicylic acid or derivatives of salicylic acid comprises methylsalicylic acid, benzoic acid, phenol, 4-hydroxybenzoic acid, acetylsalicylic acid, magnesium salicylate, choline salicylate, bismuth subsalicylate, or sulfosalicylic acid.

9. The formulation of claim 7, wherein the terpene comprises alpha pinene, linalool, beta-caryophyllene, myrcene, limonene, ocimene, terpinolene, terpineol, valencene, geraniol, humulene, phellandrene, careen, terpinene, fenchol, borneol, bisabolol, phytol, camphene, sabinene, camphor, isoborneol, menthol, cedrane, nerolidol, guaiol, isopulegol, geranyl acetate, cymene, eucalyptol, or pulegone.

10. The formulation in claim 1, wherein the effective amount of terpene is between 0.25 mg-1000 mg.

11. The formulation in claim 1, wherein the effective amount of amino acid is between 1 mg/1000 mg.

12. The formulation of claim 9, wherein the amino acids comprises theanine or GABA

13. The formulation of claim 1, wherein the formulation comprises a suspension in a liquid emulsification suitable for oral application via a pump spray.

14. The formulation of claim 1, wherein the formulation comprises the suspension is suitable for a softgel, a tablet, a capsule, a gummie, a lozenge, a chewing gum, a taffy, a vape liquid, an inhaler cartridge, an aerosol, a topical ointment, a tincture, a transdermal patch, a suppository, or an edible product.
